(19) United States
(12) Patent Application Publication
(10) Pub. No.: US 2010/0212213 A1
(43) Pub. Date: Aug. 26, 2010

(54) DETECTION DEVICE AND METHOD FOR MONITORING BED BUG INFESTATION

(76) Inventors: Joe Harold Hope, III, Mebane, NC (US); Hans Reiner Pospischil, Bergheim Fliesteden (DE)

Correspondence Address: BAYER CROPSCIENCE LP, Patent Department, 2 T .W. ALEXANDER DRIVE, RESEARCH TRIANGLE PARK, NC, 27709, US

(21) Appl. No.: 12/392,417

(22) Filed: Feb. 25, 2009

(51) Int. Cl. A01M 1/00; A01M 1/20; A01M 1/02; G01N 33/50; G01N 21/77

(52) U.S. Cl. 43/123; 43/131; 422/68.1; 422/56; 436/66

(57) ABSTRACT

A detection device and method for monitoring infestation by insects such as bed bugs is provided. A suitable device comprises a pair of plates separated by an internal spacing sized to permit entry into the detection device by one or more insects. Also disclosed is a method for readily detecting insects such as bed bugs in a multiple areas such as in multiple hotel rooms, areas where animals are housed, or the like.

### BACKGROUND

1. Field

This disclosure relates to devices and methods used to detect the presence of bed bugs and other insects that feed on the blood of warm-blooded hosts.

2. Description of Related Art

Monitoring devices are frequently used tools in pest management according to the rules of integrated pest management, for instance sticky traps with special lures for monitoring cockroach and silverfish. Monitoring-traps (in particular sticky traps), which are available in the market, are rarely accepted by bed bugs. Special devices are required for bed bug monitoring which are inexpensive, easy to handle, not visible for clients, easy to assess for room services (in hotels) or pest management professionals (PMPs), and highly attractive for bugs.

Bed bugs are small insects that feed solely on the blood of animals. The common bed bug, Cimex lectularius, is the species of bed bug that has most adapted to living with human beings. Adult bed bugs are about ¼-inch or about 6 millimeters long, 5 to 6 millimeters wide, and reddish-brown with oval, flattened bodies. The immature nymphs are similar in appearance to the adults but smaller and lighter in color.

Bed bugs do not fly but they can move very quickly over surfaces. Female bed bugs lay their eggs in secluded areas and can deposit up to five eggs per day, and as many as 500 during a lifetime. Bed bugs can survive for extended periods without feeding. For example, nymphs can survive months without feeding and the adults for can survive more than a year. As a result, infestations are not likely to be eliminated by the absence of a host in the area of concern.

Bed bugs obtain their sustenance by drawing blood through an elongated beak. They may feed on a human being for 3 to 10 minutes, although the human being is not likely to feel the bite. After the bite, the human victim often experiences an itchy welt or swelling in the area of the bite. Because some victims do not have any reaction, or only a minimal reaction to a bed bug bite, infestations may go long periods without being detected. New bed bug infestations originate from a bed bug being carried into a new area, such as by clinging to possessions which are easily transported, for example, clothing, sheets, and other items. As a consequence, living areas where the turnover of occupants is high, such as hotels or apartments, are especially vulnerable to bed bug infestations.

For the foregoing reasons, bed bugs are often difficult to detect and to eradicate. Pest management professionals (PMPs) and pesticides are typically required, necessitating removal of non-essential objects from a room, removal of bed bugs and eggs through vacuuming, and then application of pesticides to likely hiding areas. Because treatment for bed bug infestation is intrusive, disruptive to normal business operations and expensive, early detection of bed bugs and continuous monitoring for the presence of bed bugs is highly desirable. If early detection is made, appropriate steps can be taken before infestation becomes established.

What is needed, therefore, is a detection device for monitoring bed bug infestation which is attractive to bed bugs, easy to handle, discrete, and deployable across a wide range of areas, particularly in hotels and other lodging locations that experience frequent turnover of occupancy. Also needed is a method for quickly monitoring multiple rooms for bed bug infestation.

### SUMMARY

Accordingly, a detection device for monitoring bed bug infestation is provided, comprising a detection device for monitoring bed bug infestation, comprising: a first plate and a second plate, wherein the first plate and the second plate are separated by an internal spacing sized to permit entry into the detection device by one or more bed bugs; a support structure between the first plate and the second plate. The support structure may be used, for example, for maintaining the internal spacing within the device.

In one aspect, the internal spacing is between about 1 mm and about 5 mm. Optionally, the first plate and the second plate are constructed from a plastic material, advantageously a polycarbonate material. In another aspect, the first plate or the second plate or both the first plate and the second plate are at least partially transparent. In another aspect, the first plate, or the plate on which the bed bugs are expected to traverse, includes a textured surface.

According to a further aspect, there is provided a method for detecting the presence of insects, particularly bed bugs. According to yet a further embodiment, there is provided a method for monitoring the presence of insects in at least one defined area, and if insects such as bed bugs are detected, applying treatment to the area of where the insects are detected.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 is a perspective view according to one embodiment.

FIG. 2 is a front view of the embodiment of FIG. 1.

FIG. 3 is a side view of the embodiment of FIG. 1.

FIG. 4 is a top view of the embodiment of FIG. 1.

### DETAILED DESCRIPTION

The detection device may further include a bed bug attractant and/or pheromone. For example, volatile compounds isolated from bed bugs can be used for this purpose and are representative of aggregation, alarm, and/or sexual pheromones. Other potentially suitable materials include those disclosed in “Identification of the Airborne Aggregation Pheromone of the Common Bed Bug, Cimex lectularius”, Journal of Chemical Ecology, vol 34, no. 6, June 2008, which is incorporated herein by reference in its entirety. Other suitable attractants include food attractants in any form such as in solid, gel or liquid form. It is also possible to utilize carbon dioxide, methanol, methane, furan, pyridine, human perspiration, lactic acid, butyric acid, octenol, indole, 6-methyl-5-hepten-2-one, geranyl acetone, 1-dodecanol, 3-methyl-1-butanol, carboxylic acids, urea, and sebum (a component of skin oil). Suitable attractants also can include one or more harboraging agents. The detection device may also include an insecticide such as a carbamate, pyrethroid, phenylpyrazole and/or chloronicotinyl compound or any other suitable compound.

A method for detecting the presence of bed bugs is also provided. The method allows for the detection of bed bugs even when there are no bugs visible at the time of evaluation of the detection. This method takes advantage of the fact that bed bugs feed on blood and that remainders of the blood such as heme, globin, or hemeoglobin can be detected in the bugs' feces. Another aspect of this method comprises applying a fecal occult blood test to insect feces to detect the presence of blood, which is a strong indication of bed bugs as the source of the feces. Generally, any test system can be used, but it is preferred to use a detection system which allows for a visual readout, e.g., a color reaction.

In order to be able to easily apply this test to bed bug feces, it is useful to combine this method with the devices of the present disclosure which may be entered by bed bugs, and/or used by bed bugs for resting, and thus serve as a location for releasing feces. The feces can then be easily checked for the presence of blood with the above method.

Therefore, a method for detecting the presence of bed bugs is also provided, comprising: providing a detection device having an internal volume and an opening sized to permit entry into the detection device by one or more bed bugs; and detecting the presence of the bed bugs with a fecal occult blood test performed either in the device or on content of the device after removal of the same from the device.

In yet another aspect, the device described above may be prepared in such a way that a test for the presence of fecal blood can easily be performed and evaluated. To achieve that, there is preferably attached to the first plate an absorbent layer impregnated with a fecal blood detection agent, for example, a paper layer impregnated with a fecal blood detection agent. One example of a fecal blood detection agent is a guaiac-resin, which is capable of displaying a visible indicia in presence of fecal blood from the bed bug when hydrogen peroxide as developing agent is added. To allow read-out within the device, the plates of the device are preferably at least partially transparent.

Accordingly, a detection device for monitoring bed bug infestation is provided, comprising: a first plate and a second plate, wherein the first plate and the second plate are separated by an internal spacing sized to permit entry into the detection device by one or more bed bugs; a support structure between the first plate and the second plate; an optional fecal blood detection agent on an absorbent layer fixed to the first plate. The support structure between the first and second plate can be positioned in any desired manner and for any desired purpose such as for maintaining the internal spacing and/or to provoke thigmotactic behavior of the bed bugs.

Bed bugs are attracted to materials with a rough surface texture and surface porosity. Examples of materials that may be used to form the detection device itself and/or be incorporated into the detection device include wood, cardboard, corrugated cardboard, cotton, or wallpaper. Materials with a low heat transfer rate may also be suitable in some cases.

In accordance with a further embodiment, there is provided a method for monitoring the presence of insects, particularly bed bugs, in multiple discreet areas such as in hotel rooms, bedrooms, animal holding areas such as chicken houses, barn stalls, etc. In accordance with this method, a device which allows for the entry of a insect such as bed bug, is placed in each location sought to be monitored. There can be one device or hundreds or more devices depending on how many discreet areas need to be monitored separately. For example, in a hotel, a separate device could be mounted to each bed in each room in the hotel. The device could be any desired device, but advantageously is a device as disclosed herein. The device or devices are checked on a periodic basis, for example, weekly, biweekly, every 24 hours, or as desired, and if the device has evidence of insect contact such as by viewing the insect in the device, by noticing feces in the device and/or by presence of blood from insect feces, then the area where that particular device was mounted is then subjected to a treatment regime to eliminate or reduce the activity of the insect in that area.

If insects such as bed bugs are detected in one of the areas being monitored, optionally bed linens and clothing should preferably first be removed from the area. Beds should preferably be disassembled and bed bug-infested mattresses, box springs, couches or other belongings are advantageously discarded. Treatment can be with any insecticide known to be active against the insect that has infested the area.

For example, for bed bug infestation, SUSPEND SC (0.06% deltamethrin) can optionally be used as the primary liquid insecticide. Application should be thorough, targeting areas where bed bugs were found or likely to crawl or hide such as in seams, tufts and crevices of mattresses, box springs, bed frames and headboards; along and beneath baseboards; seams, tufts, and crevices of upholstered chairs, sofas and wood furniture; floor areas under beds and couches; ceiling-wall junctures; and behind wall mounts such as mirrors and picture frames. The average amount of SUSPEND SC applied per apartment on the initial service can advantageously be 0.9 gallon (range: 0.25 to 1.5 gallons). If necessary, and in most cases desirably, there can be a follow up application of the pesticide. In the follow up application, any amount of the pesticide can be used, and typically 0.25 to 0.5 gallon. Any number of follow up treatments can be done as needed or desired until eradication is accomplished. Bed bug infested/prone areas can also treated, for example, with DRIONE (silica gel plus pyrethrins) or DELTADUST (0.05% deltamethrin). Principal application sites for these dust formulations included under baseboards and carpet edges, behind outlets and switch plates, and the inner framework of couches and box springs. Additional products that can be used on a case-by-case basis include CB-80 EXTRA (0.5% pyrethrum), STERI-FAB (primarily alcohol plus D-phenothrin), and INVADER-HPX (1% propoxur).

After treatment, additional follow up inspections can be made, preferably at least on a biweekly basis. Since bed bugs are often hard to eradicate, to achieve the desired outcome, thorough inspections and repeated applications should be made and a sufficient quantity of insecticide should be used. The amount of time spent in each infested area on the initial treatment preferably ranges from 1 to 2 man hours, with each follow-up service or treatment preferably lasting at least an additional hour. Once an area has been treated, a new device should be installed in that area for future monitoring of the presence of insects. According to one embodiment, a service contract for treatment of insects such as bed bugs could include the provision of a specific number of monitoring devices which is tied to treatment if and when any of the devices detects the presence of infestation.

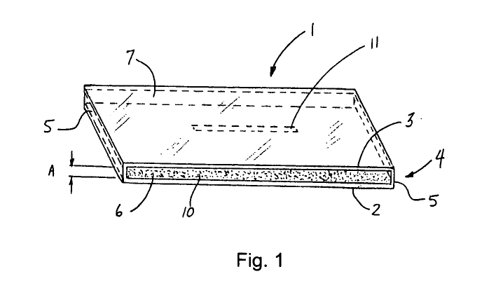

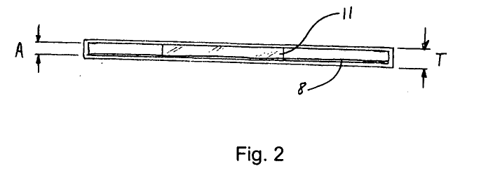

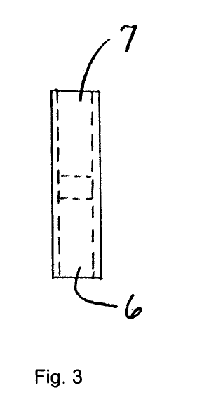

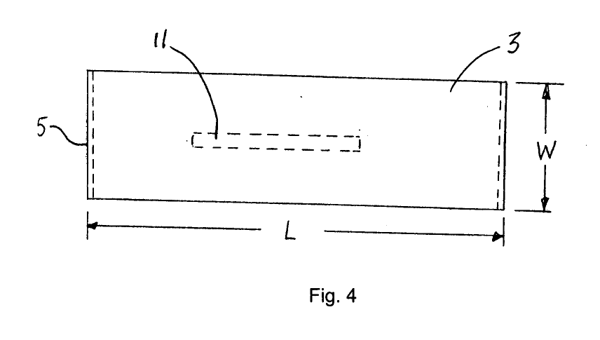

The associated FIGS. 1-4 depict exemplary configuration(s) and shape(s). Any other desired configurations and shapes for the device are also contemplated and are intended to be within the scope of the present application. FIG. 1 describes a detection device 1 for monitoring bed bug infestation according to one embodiment. As shown in FIG. 1, the detection device 1 includes a first plate 2 and a second plate 3. The first plate 2 is a surface over which the bed bugs are expected to traverse. The first plate 2 and the second plate 3 are separated by an internal spacing A, sized to permit entry into the detection device 1 by one or more bed bugs (not shown). A support structure 4 exists between the first plate 2 and the second plate 3 for maintaining the internal spacing A. Such support structure 4 can simply be side walls 5 at opposing ends of the device 1, such that entry slots 6, 7 are defined.

The internal spacing A is designed to be sufficiently large to permit bed bugs to enter the detection device 1, but still small enough to appeal to the tendencies of bed bugs to inhabit small cracks and crevices. For example, the internal spacing A may be between about 1 mm and about 15 mm, or between about 2 mm and about 13 mm, or between about 5 mm and about 11 mm, or between about 7 mm and about 9 mm. The internal spacing A may be about 1 2, 3, 4, 5, 6, 7, 8, 9, or 10 mm.

In another embodiment, a small substrate 11 (which can be for example a wood stick, plastic or any other substrate which can optionally be textured), is affixed between the first plate 2 and the second plate 3. The small substrate 11 is intended to provoke thigmotactic behavior of the bed bug. Thigmotaxis is the change in direction of locomotion of an insect made in response to a tactile stimulus, such as by touching the substrate 11. It is believed that by touching the substrate 11, the stimulus may inhibit movement, causing the bed bug to remain in close contact with the interior of the device 1.

The substrate 11 may be between about 15 mm and about 100 mm, or between about 25 mm and about 75 mm or about 50 mm long. The substrate 11 may be between about 0.5 mm and about 5 mm, or between about 0.75 and about 3 mm, or about 1 mm wide. The thickness of the substrate 11 is generally the same as the thickness T of the detection device 1.

In one embodiment, the substrate 11 is positioned parallel to the length edge of the detection device 1. In this embodiment, the substrate 11 is positioned between about 2 mm and about 20 mm, or between about 5 mm and about 17, or about 12 mm from one length edge of the detection device 1.

In another embodiment, the detection device may have multiple substrates 11, each of which may be positioned independently of each other to maximize detection of bed bugs. In another embodiment, the substrate 11 is not straight. Substrate 11 may be curved, ziz-zagged, wavy, any combination thereof or of any desired configuration.

The detection device 1 may be constructed in a wide range of sizes and shapes. Suitable ranges of width W of the detection device 1 include between about 5 mm and about 50 mm, or between about 10 mm and 40 mm, or between about 15 mm and about 35 mm, or about 25 mm. Suitable ranges of length L of the detection device 1 include between about 25 mm and about 125, or between about 50 mm and about 100 mm, or about 75 mm. Suitable ranges of thickness T of the detection device 1 include between about 1 mm and about 15 mm, or between 3 mm and about 12 mm, or between about 5 mm to about 10 mm in thickness T. Factors in determining the overall size of the detection device 1 are portability and discretion, such that the detection devices 1 can easily be placed in areas not readily visible by human occupants. The detection device 1 may be of any shape, including square, rectangular, triangular, and round. A round detection device 1 may have a radius R from about 10 mm to about 50 mm.

Optionally, the first plate 2 and the second plate 3 are constructed from a polycarbonate material. Any desired polycarbonate material could be utilized such as MAKROLON, a product marketed by Bayer MaterialScience AG, TRISTAR, a product marketed by PTS LLC, as well as any other desired material. Polycarbonate is but one option and the plates can be formed of any desired product as useful in a particular situation. In another embodiment, the first plate 2 or the second plate 3, or both, is at least partially transparent so that visual inspection of the inner surfaces of the detection device 1 may be conducted. In this manner, a user or PMP may visually detect the presence of bed bugs or whether a fecal blood detection agent indicates a reaction to fecal blood from the bed bugs.

In another aspect, the first plate 2 includes a textured surface 10, which can simply be achieved by the use of the absorbent layer 8 previously described. Such a textured surface 10 enables the bed bugs to cling to the inside of the device 1, so that when the device 1 is removed for inspection, the bed bugs are not inadvertently dislodged.

Within the detection device 1, a fecal blood detection agent may be applied to the first plate 2. One example of a fecal blood detection agent is a guaiac-resin solution, which it is capable of displaying a visible indicia in the presence of fecal blood from the bed bug. An Examples of fecal blood detection agents include the HEMOCCULT, HEMOCCULT II, HEMO-FEC, GAMMA FE-CULT, FECATEST, and CAMCO-PAC GUAIAC. After application of a developing solution (which generally comprises hydrogen peroxide) to fecal points, such fecal blood detection agents display a color change of a material in the presence of fecal blood from the bed bugs. In another embodiment, the first plate 2 includes a paper layer 8 impregnated with the fecal blood detection agent.

The detection device 1 may further optionally include a bed bug attractant or pheromone applied to the inner surfaces of the detection device 1, such as to the paper layer 8, Detection devices 1 may be refreshed with additional attractants from time to time as their effectiveness becomes attenuated after repeated use of the detection device 1.

The detection device 1 may further include an insecticide applied to the inner surfaces of the detection device 1. The insecticide may be, for example, selected from the group consisting of carbamates, pyrethroids, phenylpyrazoles, and chloronicotinyles.

One embodiment is a method for operation of the detection device 1. In this method, the detection device 1 is simply placed into areas where it is believed that bed bugs may be present, such as under mattresses, between sheets, or similar areas. Retrieval and inspection of the detection device 1 can be accomplished on a daily basis by non-technical personnel or by PMPs as needed. If the presence of bed bugs is detected in a particular detection device 1, that detection device 1 may be disposed to prevent further infestation.

The detection device 1 may be constructed of degradable components including degradable paper and one or more degradable plastics. In addition, the detection device 1 may contain degradable chemicals, such as degradable attractants, degradable insecticides, and degradable detection agents.

From the foregoing description, it can be seen that the disclosure presents a cost-effective and advantageous solution to bed bug detection and monitoring. About three to six detection devices 1 may be placed in a typical hotel room. The detection devices 1 would have a serviceable life of about 3 months to about 12 months before disposal. Hotel workers and other PMPs may place the detection devices 1 in locations that permit easy retrieval, but that are not readily discernable by room occupants.

The detection devices 1 may be attached by adhesive means or by conventional hook and loop fastening materials to bed structures or similar surfaces. In another embodiment, the detection device 1 comprises a magnetic strip.

The detection devices 1 may be manufactured individually or as a block of detection devices 1. If manufactured as a block, the individual detection devices may be separated subsequently prior to packaging. The detection devices 1 may be sealed in individual packages or in a package with a several detection devices 1.

The detection devices 1 may be manufactured as single-use or multiple-use. In addition, the detection devices 1 may be customized to include items such as a company's logo, a label that lists the room number, the date that the detection device 1 was opened or installed, the name of the hotel or motel where the detection device 1 is being used, etc. In another embodiment, the label listing the room number may be removable so that it can be catalogued or used in a reporting program.

Use of such detection devices 1 allows PMPs to realize labor savings in following up treated properties with less time than present inspection techniques. Furthermore, such PMPs can offer an added service to assure business owners that everything possible is being done to control the infestation. Similarly, hospitality industry businesses can point to such bed bug surveillance and prevention programs in defense of legal action by guests that have been affected.

The detection devices 1 described may also be used in other locations that are, or may become infested with Cimex sp. or other genera in the Cimicidae family, for example the genus Haematosiphon, or the genus Oeciacus. An example of such a location is a chicken house. In one embodiment, the detection device 1 is used in a chicken house. It is to be understood that the size and shape of detection devices 1 may be different in chicken houses or other places where poultry or animals are kept. Issues related to discretion of detection, for example smaller sizes or profiles are less important in poultry houses.

### EXAMPLES

### Example 1

This example shows that the detection devices as described are effective in attracting bed bugs even in the absence of an attractant.

Mixed populations of Cimex lectularius with different stages of nymphs and adults are put in plastic boxes (200 mm×200 mm). The population contains about 200 bed bugs. The ground of each box is covered with folded filter paper to provide shelter to the bed bugs. One detection device is put after 4 days into each box below the filter paper.

The detection devices consist of two transparent layers of MACROLON with dimensions of 25 mm×75 mm and containing an opening of 2 mm in between the layers. Two versions of the detection device are compared. One version contains a bed bug attractant. In this example, a strip of contaminated paper from a bed bug culture is used as an attractant. The other version did not contain any bed bug attractant and merely contained a small piece of filter paper.

The detection devices are assessed 1, 2, 3, 4, and 7 days. Assessment is by taking photographs. The bed bugs inside the traps are not counted to avoid disturbance.

As the table below shows, detection devices that contain an attractant attract more bed bugs than untreated detection devices. However, even the untreated detection devices attract bed bugs.

| | Treated | No. of | No. of | No. of | No. of |
|---|---|---|---|---|---|
| Device | with | bed bugs | bed bugs | bed bugs | bed bugs |
| No. | attractant? | on Day 1 | on Day 2 | on Day 3 | on Days 4 to 7 |
| 1 | Yes | 25 | 38 | 57 | 65 |
| 2 | Yes | 10 | 52 | 18 | 22 |
| 3 | Yes | 18 | 32 | 12 | 12 |
| 4 | Yes | 53 | 9 | 79 | 55 |
| 5 | Yes | 18 | 18 | 40 | 62 |
| Totals | Yes | 124 | 149 | 206 | 216 |
| 6 | No | 1 | 7 | 9 | 5 |
| 7 | No | 0 | 1 | 19 | 19 |
| 8 | No | 35 | 51 | 55 | 44 |
| 9 | No | 1 | 0 | 1 | 10 |
| 10 | No | 0 | 3 | 1 | 4 |
| Total | No | 37 | 62 | 85 | 82 |

Of course, numerous modifications and variations of the present disclosure are possible in light of the above teachings. It is therefore to be understood that within the scope of the appended claims, the disclosure may be practiced other than as specifically described herein.

Further, it is to be understood that the disclosure is not limited to the particular embodiments described below, as variations of the particular embodiments may be made and still fall within the scope of the appended claims. It is also to be understood that the terminology employed is for the purpose of describing particular embodiments, and is not intended to be limiting. Instead, the scope will be established by the appended claims.

In this specification and the appended claims, the singular forms “a”, “an”, and “the” include plural reference unless the context clearly dictates otherwise. Unless defined otherwise, all technical and scientific terms used herein have the same meaning as commonly understood to one of ordinary skill in the art to which this invention belongs.

What is claimed is:

1. A detection device for monitoring insect infestation, comprising:
a first plate and a second plate, wherein the first plate and the second plate are separated by an internal spacing sized to permit entry into the detection device by one or more insects;
a support structure between the first plate and the second plate wherein the support structure is configured to maintain internal spacing between said plates.

2. The device of claim 1, wherein the insect comprises a bed bug.

3. The device of claim 1, wherein the internal spacing is from about 1 mm to about 5 mm.

4. The device of claim 1, wherein the first plate and the second plate are constructed from a plastic material.

5. The device of claim 4, wherein the plastic material comprises polycarbonate.

6. The device of claim 1, wherein the first plate or the second plate is at least partially transparent.

7. The device of claim 1, wherein the first plate comprises a textured surface.

8. The device of claim 1, further comprising a fecal blood detection agent.

9. The device of claim 1, further comprising an absorbent material attached to the first plate, wherein the absorbent material is impregnated with a fecal blood detection agent.

10. The device of claim 9, wherein the absorbent material comprises paper.

11. The device of claim 8 or 9, wherein the fecal blood detection agent is a guaiac-resin.

12. The device of claim 8 or 9, wherein the fecal blood detection agent displays visible indicia in presence of fecal blood from a bed bug.

13. The device of claim 1, further comprising a bed bug attractant or pheromone.

14. The device of claim 1, further comprising an insecticide selected from the group consisting of carbamates, pyrethroids, phenylpyrazoles, and chloronicotinyles.

15. A method for detecting the presence of insects, the method comprising detecting occult blood in insect feces.

16. The method of claim 15, wherein the insects comprise bed bugs.

17. A method for detecting the presence of insects, comprising:
providing a detection device having an internal volume and an opening sized to permit entry into the detection device by one or more insects; and
detecting the presence of the insects with a fecal blood detection agent applied to the internal volume.

18. The method of claim 17, wherein the insects comprise bed bugs.

19. The method of claim 17, wherein the detection device is at least partially transparent.

20. The method of claim 17, wherein the internal volume includes a textured surface.

21. The method of claim 17, further comprises an absorbent layer attached to the internal volume, wherein the layer is impregnated with the fecal blood detection agent.

22. The method of claim 21, wherein the absorbent layer comprises paper.

23. The method of claim 17, wherein the fecal blood detection agent is a guaiac-resin.

24. The method of claim 17, wherein the fecal blood detection agent displays visible indicia in presence of fecal blood from the bed bug.

25. The method of claim 17, wherein the internal volume comprises a bed bug attractant or pheromone.

26. The method of claim 17, wherein the internal volume comprises an insecticide selected from the group consisting of carbamates, pyrethroids, phenylpyrazoles, and chloronicotinyles.

27. A detection device for monitoring bed bug infestation, comprising:
a first plate and a second plate, wherein the first plate and the second plate are separated by an internal spacing sized to permit entry into the detection device by one or more bed bugs;
a support structure between the first plate and the second plate, wherein the support structure is configured to promote thigmotactic behavior of bed bugs.

28. A method for monitoring the presence of insects comprising:
placing a device which allows for the entry of an insect in each location sought to be monitored,
checking each device on a periodic basis, and if any device shows evidence of insect contact therewith,
optionally, subjecting an area associated with said device showing said evidence to a treatment regime in an effort to eliminate or reduce the activity of the insect in that area.

29. The method of claim 28, wherein the insects comprise bed bugs.

30. A method for monitoring the presence of insects in a location comprising:
placing the device of claim 1 in each location sought to be monitored,
checking each device on a periodic basis, and if any device shows evidence of insect contact therewith,
optionally subjecting an area associated with said device showing said evidence to a treatment regime to eliminate or reduce the activity of the insect in that area.

31. A method of claim 30, wherein said insects comprise bed bugs.

32. A device of claim 1 that has a width from about 5 mm to about 50 mm, a length from about 25 mm to about 125mm, and a thickness from about 1 mm to about 15 mm.

33. A device of claim 1, that has a width from about 10 mm to 40 mm, a length from about 50 mm to about 100 mm, and a thickness from about 3 mm to about 12 mm.

34. A device of claim 1, that has a width from about 15 mm to about 35 mm, a length from about 50 mm to about 75 mm and a thickness from about 5 mm to about 10 mm.

35. A device of claim 32 wherein there is provided a substrate between said first and second plates, said substrate having a length of 15-100 mm.

36. A device of claim 35 wherein said substrate is oriented parallel to the length of said device.
